(19) United States
(12) Patent Application Publication
(10) Pub. No.: US 2014/0277365 A1
(43) Pub. Date: Sep. 18, 2014

(54) ULTRASOUND-GUIDED ENDOGRAFT SYSTEM

(71) Applicant: University of Rochester, Rochester, NY (US)

(72) Inventor: David L. GILLESPIE, Rochester, NY (US)

(73) Assignee: UNIVERSITY OF ROCHESTER, Rochester, NY (US)

(21) Appl. No.: 14/203,207

(22) Filed: Mar. 10, 2014

Related U.S. Application Data
(60) Provisional application No. 61/782,892, filed on Mar. 14, 2013.

(51) Int. Cl. A61F 2/962; A61B 19/00

(52) U.S. Cl. CPC A61F 2/962; A61B 19/5244
USPC 623/1.12; 623/1.11

(57) ABSTRACT

A stent delivery system can facilitate positioning of a stent within a first vessel. The system can include an elongate member, an expandable stent coupled to the elongate member, and an imaging mechanism, such as an ultrasonic probe. The imaging mechanism can be positioned distal or proximal relative to the stent or adjacent to an aperture in the stent, such that the imaging mechanism can facilitate positioning of the stent within the first vessel such that flow into an orifice of a second vessel is not blocked by the stent.

### RELATED APPLICATIONS

The present application claims the benefit of U.S. Provisional Patent Application No. 61/782,892, filed on Mar. 14, 2013, the entirety of which is incorporated by reference herein.

### BACKGROUND

Deep venous thrombosis (DVT) is the most common cause of venous outflow obstruction. Venous outflow obstruction may be either acute or chronic. Patients with acute DVT usually present with sudden onset of unilateral leg swelling. This is often painful, associated with cyanosis of the extremity, and often after prolonged immobilization or sedentary activity. Chronic venous outflow obstruction usually occurs months to years after an initial DVT. In symptomatic patients, the body's own recanalization of thrombosed veins is incomplete, and the collateral circulation is inadequate. The proximal obstruction results in distal venous hypertension, lower extremity swelling, and pain worsened after ambulation. Although venous outflow obstruction of the lower extremity may involve the entire venous system, some endovascular techniques focus treatment on thrombosis of the largest veins, namely, the inferior vena cava (IVC), common iliac vein, and external iliac veins.

Catheter-directed thrombolysis and percutaneous mechanical thrombectomy (PMT) can be important therapies in patients with acute DVT, largely replacing venous bypass surgery. Initial clinical and technical success can be achieved in most patients with acute DVT. Early thrombus removal results in relief of lower extremity venous hypertension and improved long-term patency of the venous system.

### SUMMARY

Central venous obstruction of the upper extremities is a challenging, yet increasing problem often associated with the use of chronic indwelling catheters for hemodialysis. Stenosis of the innominate vein or superior vena cava are becoming increasingly commonplace. These patients will often have failure of their upper extremity hemodialysis graft or arteriovenous fistula due to this venous outflow obstruction. Endovascular intervention with percutaneous balloon angioplasty and/or stent placement has emerged as first line treatment. Not unlike the lower extremities, there is no consensus on what the best stent configuration is to maintain the patency of the innominate vein or superior vena cava. The exact same stent configurations listed above are attempted in the chest with similar limitations and results.

There is a need for a simpler and more effective stent system suitable for use in vessel junctions, such as venous confluences or arterial bifurcations. Some embodiments disclosed herein provide methods of placing and expanding one or more stents in order to provide a reliable treatment of stenosis of vessel junctions as well as other applications. Although some embodiments are discussed in venous confluence applications, such discussion can also apply for arterial bifurcation applications. For example, while some embodiments address the clinical problem of delivering a fenestrated venous endograft safely without covering the contralateral common iliac vein orifice, some embodiments can permit delivery of an arterial endograft that does not cover branch arteries.

The placement of a stent graft in the venous system to treat iliocaval venous thrombosis is a relatively new concept. Most interventionists prefer to use an open cell stent design instead of an endograft or covered stent to treat iliocaval venous stenoses. However, an aspect of some embodiments is the realization that a fenestrated endograft can be used instead of an open cell stent design because the majority of iliocaval venous stenoses are located at the confluence of the left and right iliac veins. In order to treat this effectively, it is felt by some that stenting into the vena cava across the contralateral iliac vein is necessary. This has led to concern by some that this would lead to contralateral iliac vein thrombosis. However, according to some embodiments, this problem can be avoided by using a fenestrated endograft that is properly positioned within the confluence. For example, the fenestration in the endograft, centered over the contralateral iliac vein orifice, could allow unimpeded flow from this vein into the vena cava. Further aspect of at least some embodiments disclosed herein is the realization that aligning the fenestration with ease and reproducibility can be very difficult.

In order to address these and other challenges, some embodiments provide systems and methods for placing an endograft using an intravascular ultrasound (“IVUS”) guided delivery system. Indeed, no prior method or device has used IVUS system built into the stent delivery system, such as for a fenestrated endograft.

In some embodiments, the stent delivery system can comprise an imaging mechanism, such as an ultrasound probe or sensor, such a transducer or array of ultrasound crystals. The ultrasound probe can enable a clinician to precisely see within the vessel, junction, bifurcation, and/or confluence. For example, the clinician can see with real time imaging, the contralateral iliac vein orifice and avoid covering it. Further, in some embodiments, after using the device for delivery, the device could be re-inserted to do a final IVUS evaluation.

In some embodiments, the device can be electronically wired to a monitor where the imaging can be viewed. Further, in accordance with some embodiments, the ultrasound probe could be arranged on a dilator tip and/or on a sheath of the stent delivery system.

Thus, in some embodiments, the clinician can orient the graft fenestration accurately using the IVUS system. Additionally, the stent delivery system can comprise one or more radiopaque markers on the stent graft which can be visible under fluoroscopy. For example, such radiopaque markers can be used to help to avoid covering the contralateral common iliac vein inflow into the inferior vena cava at the iliocaval confluence.

Additionally, some embodiments also provide methods and apparatuses for treatment of traumatic injuries, such as a traumatic injury of the inferior vena cava. Conventional open repair of the traumatic injuries of the inferior vena cava still represents a surgical challenge, since it carries nigh morbidity and mortality rates close to 100% in emergency setting. Surgical techniques required the total mobilization of the liver, a skill not possessed by most general surgeons. Various publications address the use of stent grafts and the challenges associated with traumatic injuries of the vena cava and other vasculature, including: Jung, M., et al., Apparatus and Method for the Intravascular Guided Placement of a Vena Cava Filter, 2002: USA; Petersen, B., et al., Intravascular US-Guided Direct Intrahepatic Portacaval Shunt With a PTFE-Covered Stent-Graft: Feasibility Study in Swine and Initial Clinical Results, J. Vase Interv. Radiol., 2001, at 12(4): p. 475-86; Neglen, P., et al., Stenting of the Venous Outflow in Chronic Venous Disease: Long-Term Stent-Related Outcome, Clinical, and Hemodynamic Result, J. Vase Surg., 2007, at 46(5): p. 979-90; Gillespie, D. L. and D. Mix, Fenestrated Endograft, 2011: USA; Jan, W. A., A. Samad, and R. Anwar, Mortality and Morbidity of Abdominal Inferior Vena-Caval Injuries, J. Coll. Physicians Surg. Pak., 2004, at 14(10); p. 622-5; Tyburski, J. G., et al., Factors Affecting Mortality Rates in Patients with Abdominal Vascular Injuries, J. Trauma, 2001, at 50(6): p. 1020-6; Phelan, R. J. P. Hunt, and Y. Z. Wang, Retrohepatic Vena Cava and Juxtahepatic Venous Injuries, South Med. J., 2001, at 94(7): p. 728-31; Hansen, C. J., et al., Abdominal Vena Caval Injuries: Outcomes Remain Dismal, Surgery, 2000, at 128(4): p. 572-8; Chen, C. J., et al., Factors Determining Operative Mortality Of Grade V Blunt Hepatic Trauma, J. Trauma, 2000, at 49(5): p. 886-91; Rosengart, M. R., et at, Prognostic Factors in Patients with Inferior Vena Cava Injuries, Am. Surg., 1999, at 65(9): p. 849-55 (see discussion at p. 855-6); Ombrellaro, M. P., et al., Predictors of Survival After Inferior Vena Cava Injuries, Am. Surg., 1997, at 63(2): p. 178-83; Ai-jun, L, et al., Management of Retrohepatic Inferior Vena Cava Injury During Hepatectomy for Neoplasms, World J. Surg., 2004, at 28(1): p. 19-22; Khaneja, S. C., et al., Management of Pentrating Juxtahepatic Inferior Vena Cava Injuries Under Total Vascular Occlusion, J. Am. Coll. Surg., 1997, at 184(5): p. 469-74; Weber, S., et al., Management of Retrohepatic Venous Injuries with Atrial Caval Shunts, AORN J. 1996, at 64(3): p. 376-7, 380-2; Picard, E., et al., Use of Active Shunt for Surgical Repair of Intrapericardial Inferior Vena Caval Injury, Ann. Thorac. Surg., 1995, at 59(4): p. 997-8; Porta, R. M., et al., An Experimental Model for the Treatment of Lethal Bleeding Injury to the Juxtahepatic Vena Cava with Stent Graft, J. Trauma, 2006, at 60(6): p. 1211-20; Fujisawa, Y., et al., Aortocaval Fistula After Endovascular Stent-Grafting of Abdominal Aortic Aneurysm, J. Cardiovasc. Surg. (Torino), 2009, at 50(3): p. 387-9; Waldrop, J. L., Jr., B. W. t. Dart, and D. E. Barker, Endovascular Stem Graft Treatment of a Traumatic Aortocaval Fistula, Ann. Vasc. Surg., 2005, at 19(4): p. 562-5; de Naeyer, G. and L Degrieck, Emergent Infrahepatic Vena Cava Stenting for Life-Threatening Perforation, J. Vase Surg., 2005, at 41(3): p. 552-4; Castelli, P., et al., Emergency Endovascular Repair For Traumatic Injury of the Inferior Vena Cava, Eur. J. Cardiothorac Surg., 2005, at 28(6): p. 906-8; Watarida, S., et al., Fenestrated Stent-Graft for Traumatic Juxtahepatic Inferior Vena Cava Injury, J. Endovasc. Ther., 2001, at 9(1): p. 134-7, the entireties of which are incorporated herein by reference. An aspect of some embodiments is the realization that a minimally invasive endovascular solution to this highly mortal condition is needed.

The use of stent grafts to treat injuries of the vena cava was first shown to be feasible in animal models. Human applications of stent grafting to control injuries to the infrarenal IVC have been reported. However, an aspect of some embodiments is the realization that a challenge to successfully controlling injuries to the infrarenal IVC is that the drainage of the liver occurs through the hepatic veins which drain in the retrohepatic cava. Accordingly, some embodiments herein recognize that one cannot simply cover the entire retrohepatic cava without dire consequences including death from liver failure.

Accordingly, some embodiments provide for methods and apparatuses of placing a stent within a main vessel to provide coverage or treatment of an injury while still allowing flow through the main vessel to all, a majority, or a plurality of branch vessels. The injury can be a traumatic injury.

For example, some embodiments provide for methods and apparatuses of placing a fenestrated endograft within the infrarenal IVC such that the traumatic injury is covered while an aperture or fenestration allows flow through the main vessel to all, a majority, or a plurality of the hepatic veins extending from the infrarenal IVC.

In some embodiments, the stent can have a generally cylindrical, non-tapering shape. In some embodiments, the stent can have a diameter from about 15 mm to about 27 mm in size. For example, the diameter can be from about 18 mm to about 24 mm in size. Further, the diameter can be from about 20 mm to about 22 mm in size.

Additionally, according to some embodiments, the stent can comprise one or more hooks or engagement features to secure the stent in place within the vessel in order to avoid stent migration.

In accordance with some embodiments, a system is provided for positioning a stent. For example, the stent can be placed at a vessel junction, a bifurcation, or to extend along a traumatic injury to the vessel. The junction can comprise a venous confluence or an arterial bifurcation. Further, in some embodiments, the stent can be configured to permit flow to a second vessel (e.g., one or more branch vessels or an iliac vein) that bifurcate from or extend off of the first vessel (e.g., a parent vessel).

The system can comprise an elongate member, an expandable stent, and an ultrasound probe. The stent can be coupled to the elongate member in a collapsed configuration. The stent can have a sidewall aperture. The ultrasound probe can be positioned adjacent the aperture. The probe can be configured to emit an ultrasound beam radially outwardly from the aperture while the stent is in the collapsed configuration. For example, the probe can be configured to emit the ultrasound beam along an axis extending through the aperture. In some embodiments, the probe can be configured to emit the beam in a direction substantially normal to the aperture. Further, the probe can be configured to emit the beam radially outwardly from a location that is longitudinally and circumferentially aligned or adjacent to the aperture. Furthermore, in some embodiments, the probe can be configured to emit the beam radially outwardly through the aperture. Further, when the elongate member and stent are advanced through a first vessel, the probe can emit the beam to facilitate placement of the aperture across an orifice of a second vessel.

Additionally, the system can also be configured such that the ultrasound probe is coupled to the elongate member and positioned distal to the stent distal end. The ultrasound probe can also be coupled to a dilator tip. The elongate member can comprise the dilator tip.

The system can be configured such that the ultrasound probe is positioned radially within the stent. The ultrasound probe can also be coupled to the elongate member. The ultrasound probe can comprise a crystal element.

The probe can be configured to emit an ultrasound beam within a first vessel to facilitate placement of the stent distal end spaced apart from an orifice of a second vessel. Further, the ultrasound probe can be configured to emit the beam in a radial direction.

In some embodiments, the elongate member comprises a catheter. Thus, at least a portion of the elongate member can comprise a lumen. However, the elongate member can also comprise a solid core element.

Further, the system can also comprise a sheath extending over the stent. In such embodiments, the ultrasound probe can be coupled to the sheath.

In some embodiments, the stent can be coupled to the elongate member in a collapsed configuration and subsequently released into an expanded configuration when properly positioned at the treatment site relative to surrounding vasculature.

In some embodiments, methods for implanting a stent in a first vessel (e.g., a parent vessel) are also provided. For example, a method can comprise advancing, into a first vessel, a delivery member carrying an expandable stent having an aperture formed in a sidewall of the stent. The first vessel can be imaged with an ultrasound probe coupled to the delivery member to locate a second vessel (e.g., a branch vessel). For example, the imaging can take place after the stent is positioned within the first vessel using an ultrasound probe positioned on an elongate member supporting the stent. After the imaging is completed, the stent can be positioned within the first vessel such that the aperture is longitudinally and rotationally aligned with an orifice of a second vessel. Thereafter, the stent can be expanded such that the aperture opens to the orifice of the second vessel.

In some embodiments, wherein the first vessel forms a junction with the second vessel, a second expandable stent can be positioned at the junction. The second stent can have a first section, a second section, and a second aperture formed in a sidewall of the second stent. The second stent can be expanded at the junction, such that (1) the first section extends through the aperture and into the second vessel, (2) the second aperture opens to the primary vessel, and (3) the second section is positioned within the primary vessel. The second stent can be positioned such that the second aperture is aligned with a lumen of the stent, to maintain fluid communication through the stent.

A stent used in any of the methods disclosed herein can be an endograft stent. The stent aperture can have a curved perimeter formed by a circle projected against a cylinder. The stent aperture can extend about an entire circumference of the stent.

In some embodiments, the aperture can comprise a cross-sectional dimension that is substantially equal to a cross-sectional dimension of an outer cross-sectional dimension of the second stent first portion. Patency of the aperture can be maintained by expanding the second stent within the aperture.

In some embodiments, the central portion can be coupled to a fabric that extends along a perimeter of the central portion, and the aperture can be formed in the fabric.

The imaging can comprise locating a plurality of second vessels, and the plurality of second vessels can comprise a plurality of orifices. Further, the expanding can comprise orienting the stent such that the (i) the aperture opens to the plurality of orifices and (ii) the first portion extends along a traumatic injury of the first vessel.

Further, the first vessel can be an inferior or superior vena cava. Further, the second vessel orifice can open to a hepatic vein. The hepatic vein can comprise at least one of the right hepatic vein, the middle hepatic vein, or the left hepatic vein. The hepatic vein can also comprise a short hepatic vein.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 illustrates the medical condition of stenosis of the iliocaval confluence.

FIGS. 2A-2C illustrate various methods of stenting an iliocaval confluence.

FIGS. 3A-3B illustrate embodiments of a stent or fenestrated endograft.

FIG. 4 is a schematic illustration of a stent delivery system placed in a patient, according to some embodiments.

FIGS. 5A-5F illustrate a method of utilizing a fenestrated endograft to stent an iliocaval confluence according to certain embodiments of this disclosure;

FIG. 6 illustrates the further placement of stents in the inflow and outflow veins according to certain embodiments of this disclosure;

FIG. 7 illustrates an example of unilateral iliac venous obstruction;

FIGS. 8A-8B illustrate the current “hanging stent” method of treating unilateral iliocaval stenosis and a typical failure pattern;

FIGS. 9A-9B illustrate the current “understenting” method of treating unilateral iliocaval stenosis and a typical failure pattern;

FIGS. 10A-10B illustrate the current “extended stenting method” of treating unilateral iliocaval stenosis and a typical failure pattern;

FIGS. 11A-11B illustrates the use of a fenestrated endograft to treat stenosis at the confluence of the superior vena cava and the left and right brachiocephalic veins;

FIG. 12 illustrates the use of a fenestrated endograft to treat stenosis at the confluence of the superior vena cava and the left and right brachiocephalic veins.

FIG. 13 illustrates a schematic representation of the liver and inferior vena cava, along with related hepatic veins.

FIGS. 14A-14B illustrate schematic representations a trauma or injury to the inferior vena cava and placement of a stent or endograft, according to some embodiments.

FIGS. 15A-15B illustrate another schematic representations a trauma or injury to the inferior vena cava and placement of a stent or endograft, according to some embodiments.

### DETAILED DESCRIPTION

In the following detailed description, numerous specific details are set forth to provide an understanding of the subject technology. It will be apparent, however, to one ordinarily skilled in the art that the subject technology may be practiced without some of these specific details. In other instances, well-known structures and techniques have not been shown in detail so as not to obscure the subject technology. Like components are labeled with identical element numbers for ease of understanding.

The terms “stentgraft,” “endograft,” “fenestrated stentgraft,” “fenestrated endograft,” and “fenestrated venous stentgraft” have been used herein to identify the same item and should be considered equivalent, including combinations and further variations of these terms. The term “stent” is considered to cover all forms of expandable elements that retain their shape after expansion and are suitable for use in the vessels of the human body, e.g., veins and arteries. The term “graft” is considered to cover all forms of artificial structures that replace or supplement natural elements of the body, e.g., tubular fabric structures. Various aspects of the present inventions also relate to U.S. application Ser. No. 13/396,523, filed Feb. 14, 2012, and U.S. Provisional Application No. 61/442,633, filed Feb. 14, 2011, the entireties of each of which are incorporated by reference herein.

Stenosis, which is an abnormal narrowing, of a blood vessel may occur in the thrombosed vein. Clinical experience has shown that venous stenoses of the lower extremity rarely respond to angioplasty alone, and therefore, stenting is recommended. FIG. 1 illustrates a confluence 100 in a venous system 110, between the common iliac vein 112, the contralateral iliac vein 114, and the inferior vena cava 116. Stenting at such confluences to address narrowing 130 or lesions 132 in the vessels or traumatic injuries is particularly challenging. Stents may be nitinol or stainless steel.

Techniques that have been attempted for stenting of venous confluences include (i) placement of two stents 210, 212 side by side in a “double barrel” arrangement across a vessel confluence 214, as seen in FIG. 2A, (ii) inverted Y-stenting of a stent through a fenestra (e.g., a window) created through the side braiding of a stent 230 placed previously across the confluence 232 using a second stent 234, as shown in FIG. 2B, and (iii) apposition of a stent 250 as close as possible to another stent 252 previously placed across the confluence 254, as illustrated in FIG. 2C, leaving a small area unsupported between the stents.

However, the placement of a stent graft in the venous system to treat iliocaval venous thrombosis is a relatively new concept. Most interventionists prefer to use an open cell stent design instead of an endograft or covered stent to treat iliocaval venous stenoses.

An aspect of some embodiments is the realization that the majority of iliocaval venous stenoses are located at the confluence of the left and right iliac veins. However, embodiments herein recognize the concern of crossing or blocking the contralateral iliac vein, which could lead to contralateral iliac vein thrombosis. Accordingly, some embodiments herein disclose the use of a fenestrated endograft that is properly positioned within the confluence.

For example, a fenestration in an endograft, centered over the contralateral iliac vein orifice, could allow unimpeded flow from this vein into the vena cava. Further aspect of at least some embodiments disclosed herein is the realization that aligning the fenestration with ease and reproducibility can be very difficult.

In order to address these and other challenges, some embodiments provide systems and methods for placing an endograft using an intravascular ultrasound (“IVUS”) guided delivery system. Indeed, no prior method or device has used IVUS system built into the stent delivery system, such as for a fenestrated endograft.

In some embodiments, the stent delivery system can comprise an imaging mechanism, such as an ultrasound probe or sensor, such a transducer or array of ultrasound crystals. The ultrasound probe can enable a clinician to precisely see within the vessel, junction, bifurcation, and/or confluence. For example, the clinician can see with real time imaging, the contralateral iliac vein orifice and avoid covering it. Further, in some embodiments, after using the device for delivery, the device could be re-inserted to do a final IVUS evaluation.

In some embodiments, the device can be electronically wired to a monitor where the imaging can be viewed. Further, in accordance with some embodiments, the ultrasound probe could be arranged on a dilator tip and/or on a sheath of the stent delivery system.

Thus, in some embodiments, the clinician can orient the graft fenestration accurately using the IVUS system. Additionally, the stent delivery system can comprise one or more radiopaque markers on the stent graft which can be visible under fluoroscopy. For example, such radiopaque markers can be used to help to avoid covering the contralateral common iliac vein inflow into the inferior vena cava at the iliocaval confluence.

FIGS. 3A-3B illustrate an exemplary stent, stentgraft, or fenestrated endograft 500 according to certain embodiments of this disclosure. FIG. 3A illustrates the endograft 500 in an expanded configuration, such as after placement or deployment in a patient. In some embodiments, the endograft 500 comprises a stent frame 505 which may be formed of three stent members 501, 502, and 503. The stent frame can 505 may be made of expandable materials such as a metal wire. The stent frame may be made of Nitinol. The stent frame 505 may comprise other biocompatible materials that are expandable or that have shape-memory properties. The stent frame 505 may be expandable, for example, radially, from a collapsed configuration, or collapsible from an expanded configuration.

In some embodiments, these three stent members 501, 502, and 502 may be arranged and sutured in series to form a proximal end portion 501, a central portion 502, and a distal end portion 503, respectively. As shown, the expanded stent frame 505 defines a tubular or cylindrical stent lumen 506 therein. The stent lumen 506 provides a fluid communication or fluid passageway throughout the proximal end portion 501, the central portion 502, and the end distal portion 503 of the stent frame 505. In some embodiments, the proximal and distal portions 501 and 503 may be of an open-cell stent design, while the central portion 502 may uniquely include at least one curved segment, for example, a series of sinusoidal waves, extending along a perimeter thereof or the lumen 506 defined by the stent frame 505.

The central portion 502 can include a curved, elongate member 502A. This elongate member 502A can be integrally formed with the proximal end portion 501 and/or the distal end portion 503 of the stent frame 505. The elongate member 502A may otherwise be a separate member that is coupled to the proximal end portion 501 and/or the distal end portion 503 of the stent frame 505. In some embodiments, the elongate member 502A can be a separate member that is not directly coupled to the proximal end portion 501 and/or the distal end portion 503 of the stent frame 505. Various flexibilities are available for the central portion 502 by adjusting the elongate member 502A in size, shape, connectivity to portions of the stent frame 505, etc. In some embodiments, in which greater flexibility is desired, the elongate member 502A extending in the central portion 502 is not directly connected to the proximal end portion 501 or distal end portion 502 of the stent frame 505.

The endograft 500 can comprise a fabric. For example, the stent frame 505, or at least the central portion 502 of the stent frame 505, can be coupled to a fabric 504 such as a thin-walled Dacron graft with an aperture, an opening, or a fenestration 504A therein. The fabric 504 may be a nonporous material, such as polymer sheets and films. In the example as shown in FIG. 3A, the fenestration 504A is aligned within the central portion 502.

The fenestration 504A may be configured to fit a body lumen of the patient. For example, the fenestration 504A may have a cross-sectional dimension substantially equal to a cross-sectional dimension of an outer cross-sectional dimension of the distal portion 503. The similar sizing of the aperture 504A and the cross-section dimension of an outer cross-section dimension of the distal portion 503 facilitates deployment and functionality of the stent graft system 500 when used with a second stent graft 610 as illustrated in FIG. 5F.

In some embodiments, the aperture 504A can comprise an oval shape, and in some embodiments, the aperture 504A comprises at least a portion having an oval shape. The aperture 504A may comprise a shape that is defined by a circle projected onto a cylinder wall along a projection line that passes through a central axis of the cylinder and at an angle relative to a central axis of the cylinder.

The curved segment 502A of the central portion 502 can extend along at least a part of the perimeter of the fenestration 504A. The curved segment 502A may be in contact or tied with the perimeter of fenestration 504A at three points so as to extend or edge about at least ¼ of the perimeter of the fenestration 504A; and thereby provides a bias or force to keep the fenestration 504A open. The curved segment 502A may have a radius of curvature that is less than 10% than a radius of curvature of the ¼ of the perimeter of the fenestration 504A. The radius of the curvature of the curved segment 502A may be as small as less than about 5% than the radius of curvature of the ¼ of the perimeter of the fenestration 504A.

The endograft 500 may further comprise a metal or radio-opaque mark, e.g., an asymmetric symbol or character such as a letter “C” formed from metal such as gold sewn or adhered onto the fabric 504 adjacent to the fenestration 504A. Thereby, radiation such as x-ray imaging can be used during the insertion procedure to facilitate orienting the fenestration 504A in the desired direction and location.

FIG. 3B shows the endograft 500 of FIG. 3A in a compressed configuration such that it can be inserted within a sheath for introduction through a blood lumen, vessel, or other channel in the human body. It is appreciated that, apart from the radial expansion, the stent frame 505 may also be configured to be expandable along various orientations or directions, for example, longitudinally or laterally. The configuration as shown in FIG. 3B allows the endograft 500 to be loaded into a hydrophilic sheath, for example, the sheath 600 as shown in FIG. 5A, with a tapered tip to facilitate insertion into a body lumen of a patient.

In certain embodiments, the stent frame 505 can, when fully expanded, come in diameters of about 5-30 mm. The fully expanded diameter of the stent frame 505 may be about 12-24 mm. The fenestration 504A can increase in size along with graft diameter in sizes about 14-24 mm. In other embodiments, the diameters are small and larger. In this embodiment, all of the component stents are 30 mm in length and the overall graft length would be standardized at 90 mm. In certain embodiments, the endograft 500 is fully expanded when the graft material is under tension around the entire circumference of the tubular graft. In the collapsed configuration, the stent frame 505 may have a diameter of about 2-4 mm, or between about 3-4 mm, for example. In some embodiments, the diameter of the stent frame 505, when in the collapsed configuration, is less than about 2 mm, and in some embodiments, the diameter of the stent frame, when in the collapsed configuration, is greater than about 5 mm.

FIG. 4 illustrates a schematic representation of a stent delivery system 550, according to some embodiments. The stent delivery system 550 can comprise a guidewire 552, a catheter assembly 554, a monitor 556, a stent assembly 558, and an imaging mechanism 560. The catheter assembly 554 can comprise an elongate member, such as a solid core wire or catheter. As shown, the stent delivery system can be used to deliver a stent or endograft to a vessel within a patient's body. Further, the imaging mechanism 560 of the delivery system 550 can allow the clinician to locate the stent assembly 558 relative to surrounding vasculature in order to achieve a desired position within the vessel.

In some embodiments, the imaging mechanism 560 can comprise an intravascular ultrasound (“IVUS”) guided delivery system. For example, the IVUS can comprise one or more ultrasound probes or sensors. The probes can comprise one or more ultrasound crystal elements. The probe(s) can be arranged on the catheter assembly 554 and/or stent assembly 558. Further, in some embodiments that in corporate a sheath into the catheter or stent assemblies 554, 558, the probe(s) can be arranged on the sheath. The probe(s) can be mounted or attached to such components and in electrical communication another component in order to provide data regarding the vasculature. For example, the probe(s) can provide data to the monitor 556, which can be used to provide a real-time display of the configuration of the vasculature and/or the position of the stent, catheter assembly 554, and/or stent assembly 558 within the vasculature.

For example, FIGS. 5A-5F illustrate a method of utilizing an IVUS-guided stent system to deliver a fenestrated endograft to stent an iliocaval confluence, according to some embodiments.

FIG. 5A illustrates an application of the venous graft with a fenestration or aperture similar to that of the endograft 500 as shown in FIG. 3A, for example. After predilation of the venous occlusion or stenosis, such as with an angioplasty balloon, the endograft 500 carried within a sheath 600 is delivered into position over a guidewire 601. Before being deployed in the desire location, the endograft 500 may be in the compressed configuration as shown in FIG. 3B. The metal, such as gold marker C attached next to the fenestration 504A may be radiopaque, such that a cannulation gate orientation may be indicated by the C marker to line up anterior/posterior and left/right toward contralateral venous lumen 60.

In some embodiments, the expandable stent frame 505 has a diameter, when in the expanded configuration, of between about 5 mm to about 30 mm. In certain embodiments, the stent frame 505 has a diameter, when in the expanded configuration, of between about 12 mm to about 24 mm. In some embodiments, the diameter of the stent frame, when in the expanded configuration, is less than about 5 mm, and in some embodiments, the diameter of the stent frame 505, when in the collapsed configuration, is greater than about 30 mm.

The delivery system can be configured with at least one imaging mechanism arranged thereon. As illustrated in FIG. 5A, a dilator tip 602 of the delivery system can be configured with an imaging mechanism arranged thereon. For example, the imaging mechanism can be coupled to the dilator tip 602, a sheath, an elongate core member (such as a guide wire or catheter), or other suitable location on the delivery system. The imaging mechanism can comprise an IVUS system, as discussed herein. The imaging mechanism can comprise a single imaging mechanism positioned adjacent to an aperture of the endograft, at a position distal to a distal end of the endograft, and/or at a position proximal to a proximal end of the endograft.

The imaging mechanism can comprise an imaging mechanism 590, such as an ultrasound probe or sensor, that can be used to detect the presence of an orifice of a branch vessel 100 or whether the dilator tip and stent member 501 are positioned adjacent to a vessel wall 102. In this manner, the imaging mechanism 590 can detect whether the stent member 501 would obstruct an orifice of a branch vessel 100. If such an obstruction is possible, the clinician can then adjust the longitudinal position of the endograft 500 within the contralateral lumen 60.

In some embodiments, the delivery system can also comprise a second imaging mechanism 592, such as an ultrasound probe or sensor. The second imaging mechanism 592 can be located proximally relative to the stent. Further, in embodiments where the second imaging mechanism 592 is used with a first imaging mechanism 590, the second imaging mechanism 592 can be located proximally relative to the first imaging mechanism 590. Similar to the use of the imaging mechanism 590, the second imaging mechanism 592 can also be used to ascertain the presence and position of orifices of branch vessels, such as the common iliac vein orifice 62. Through the use of the second imaging mechanism 592, the aperture or fenestration 504A of the endograft 500 can be rotationally and longitudinally aligned with the vein orifice 62.

For example, as shown in FIG. 5A, the second imaging mechanism 592 can be positioned adjacent to an aperture 504A of the endograft 500 when the endograft 500 is in a collapsed position within the sheath 600. In some embodiments, the endograft 500 can also be positioned over an elongate member, such as a guide wire or catheter. As illustrated, the second imaging mechanism 592 can be configured to emit a beam or signal, such as an ultrasound beam through the aperture 504A in order to facilitate discovery of the location of branch vessels.

As shown in FIG. 5B, when the sheath 600 is positioned at the desired location in the venous lumen 60, the outer sheath 600 of delivery system is pulled back to allow the self-expanding endograft 500 to be deployed in a position adjacent to the contralateral common iliac vein orifice 62. As shown, the stent members 501 and 502 released from the sheath 600 start to expand against the walls of the venous lumen 60, followed by the self expansion of the stent member 503 as the sheath 600 is pulled further away as shown in FIG. 5C. When the sheath 600 is completely pulled away from the endograft 500, the angioplasty balloon or dilator tip 602 with an appropriate size may be used to further dilate the endograft 500.

In some embodiments, the endograft 500 can also be balloon-expandable, and the system can comprise a balloon catheter or other dilation mechanism for expanding the endograft 500 when positioned at the proper location within the vessel.

As shown in FIG. 5C, after the endograft 500 has expanded, the fenestration 504A may be positioned across the contralateral common iliac vein orifice 62 to allow a contralateral stent, for example, the stent 610 as shown in FIG. 5E to be inserted from the contralateral venous lumen 60A into the endograft 500.

FIG. 5D illustrates the endograft 500 in place after the angioplasty. As shown, the fenestration 504A is aligned with the contralateral common iliac vein orifice 62 to define a contralateral port. Once the endograft 500 is in place, a second guidewire 602 can be placed at the site and passed into the contralateral venous lumen 60A.

FIG. 5E depicts the second stentgraft 610 being inserted through the fenestration 504A of the endograft 500 into the contralateral iliac after cannulation of the contralateral gate. In some embodiments, the second stentgraft 610 may be a fenestrated venous stentgraft with a structure similar to the endograft 500. The second stentgraft 610 may be deployed in a similar manner for deploying the endograft 500. In particular, an imaging mechanism 594, and one or more other imaging mechanisms (not shown), can be used to locate the orifices of branch vessels in order to position the stentgraft 610 within the contralateral venous lumen 60A. Accordingly, an aperture 604A of the second stentgraft 610 can be aligned relative to the lumen 60 within the endograft 500. In some embodiments, a perimeter of the aperture 604A can be substantially coextensive with a perimeter of the lumen 60 of the endograft 500. Additionally, a distal end or second member 613 of the second stentgraft 610 can be positioned within the distal member 503 of the endograft 500. For example, the distal end or second member 613 of the second stentgraft 610 can extend within the distal member 503 of the endograft 500 and be expanded against an inner surface of the distal member 503. In some embodiments, the second member 613 and the distal member 503 can be substantially coextensive along the interior of the vessel.

After the second stentgraft 610 is deployed and extends through the venous lumen 60, as shown in FIG. 5F, an angioplasty balloon 612 can be inserted through the second stentgraft 610 to further dilate the second stentgraft 610. Thereby, the second stentgraft 610 has a portion extending through at portions of the central portion 502 and the proximal end portion 501 of the endograft 500, and another portion extending along the contralateral venous lumen 60A. The portion extending through the portions of the endograft 500 may expand towards the expanded stent frame 505, while the portion extending through the contralateral venous lumen 60A may extend against the sidewall of the lumen 60A. Thereby, a fluid communication or passageway is established.

As shown in FIG. 6, in some embodiments, once the endograft 500 and the second stentgraft 610 are deployed and secured at the desired location, for example, the bifurcated venous lumens 60 and 60A, the iliac vein confluence is reconstructed. Additional stents 700 may further be installed in the inflow and/or outflow of the venous lumen 60 and/or 60A using standard techniques.

Cases of unilateral iliac venous obstruction are more common than bilateral cases and may not be associated with a thrombotic state. FIG. 7 shows an example of unilateral iliac venous obstruction 750. This nonthrombotic iliac vein stenosis 750 may be treated with venoplasty and stenting techniques similar to those used in thrombotic cases. FIGS. 8A and 8B show an exemplary configuration, that is, a “hanging stent 900,” operative to treat the unilateral iliac venous obstruction. As shown in FIG. 8A, the “hanging stent” configuration centers a single stent on the lesion but does not extend the stent into the vena cava. This theoretically uses the strongest part of the stent in the most difficult part of the lesion and spares the contralateral side. These stents are often undersized and associated with stent thrombosis involving both common iliac veins as seen in FIG. 8B.

FIGS. 9A-9B illustrate an “understenting” method of treating unilateral iliocaval stenosis. “Understenting” the lesion is often performed as shown in FIG. 9A for fear of causing problems with the contralateral iliac vein. Unfortunately this technique may center the weakest portion of the stent 1000 in the worst part of the lesion. As a consequence, these stents can thrombose due to narrowing at this distal end as shown in FIG. 9B.

FIGS. 10A-10B illustrate an “extended stenting” method of treating unilateral iliocaval stenosis. Extending the stent 1100 into the vena cava is often used to treat unilateral iliac vein lesions. As shown in FIG. 10A, this stent configuration centers the stent 1100 on the lesion and allows for the use of larger stents to help the stent 1100 be adjacent to the wall of the vena cava. This technique, however, by definition places the stent 1100 across the orifice of the contralateral common iliac vein and can lead to contralateral iliac vein thrombosis, as shown in FIG. 10B.

According to some embodiments, the imaging methods and systems disclosed herein can be used to improve the positioning of a stent or endograft across lesions and to address conditions or obstructions, such as those illustrated in FIGS. 7-10B. For example, FIGS. 11A-11B show the application of the fenestrated endograft, for example, the endograft 500 as shown in FIGS. 3A-3B, to the unilateral iliac venous obstruction. As shown in FIG. 11A, and similar to the methods discussed above with reference to FIGS. 5A-5F, as the fenestration 504 of the endograft 500 can be aligned with the contralateral common iliac vein orifice 62, use of the endograft 500 has the advantages of delivering the strongest portion of the stentgraft over the lesion while avoiding obstruction the contralateral iliac vein. This allows aggressive treatment of the lesion at the iliocaval junction and leads to a lower incidence of this complication. It would also allow extension of the conventional open cell stents to treat the remaining portion of the lesion.

FIG. 11B illustrates the placement of additional stents 1150, which can be delivered according to embodiments of the methods discussed above with reference to FIGS. 5A-5F, to cover the entire lesion according to certain embodiments of this disclosure. Once the endograft 500 is delivered, the interventionist can use typical stenting techniques to insure that the entire lesion is covered with the strongest portions of the stent maintaining the largest lumen possible. The fenestrated venous endograft 500 can be used in the less complex cases of nonthrombotic iliac vein stenosis. While it provides the advantages of delivering the strongest portion of the stentgraft over the lesion, the endograft 500 would not obstruct the contralateral iliac vein orifice and leads to a lower incidence of complication. It also allows extension of the conventional open-cell stents to treat the remaining portion of the lesion.

Central venous obstruction of the upper extremities has been a challenging and increasing problem. This problem is often associated with the use of chronic indwelling catheters for hemodialysis. While thrombosis of the subclavian vein should have decreased due to the technique of using jugular venous insertion, stenosis of the innominate vein or superior vena cava are becoming increasingly commonplace. Patients with this problem often have the failure of the upper extremity hemodialysis graft or arteriovenous fistula due to this venous outflow obstruction. Endovascular intervention with percutaneous balloon angioplasty and/or stent placement has emerged as first line treatment. Accordingly, embodiments of the methods and systems disclosed herein, such as the fenestrated endograft as described above can be used to treat this problem. FIG. 12 illustrates the use of a fenestrated endograft, which can be delivered according to embodiments of the methods discussed above with reference to FIGS. 5A-5F, to treat stenosis at the confluence of the superior vena cava and the left and right brachiocephalic veins.

Features of the endograft(s) used in embodiments of the methods and systems disclosed herein can be configured according to the features and/or dimensions disclosed in copending U.S. patent application Ser. No. 13/396,523, filed Feb. 14, 2012, the entirety of which is incorporated herein by reference.

In some embodiments, a second stent graft can be configured to extend a portion of the stent graft through the aperture, such that a first portion of the second stent graft is extending within and along a portion of the first stent graft and a second portion of the second stent graft is extending through the aperture and into a separate vessel than within which the first stent graft extends. Illustrated examples of this are provided, for example, in FIGS. 5A-6. A second aperture, within the second stent graft, can be configured to be aligned with the lumen of the central portion of the first stent graft so as to maintain substantially unobstructed fluid communication through the first stent graft as well as the second stent graft. Further, as noted above, the methods and systems disclosed herein can be used at vessel junctions, such as arterial bifurcations or venous confluences.

Additionally, some embodiments also provide methods and apparatuses for treatment of traumatic injuries, such as a traumatic injury of the inferior vena cava. Conventional open repair of the traumatic injuries of the inferior vena cava still represents a surgical challenge, since it carries nigh morbidity and mortality rates close to 100% in emergency setting. Surgical techniques required the total mobilization of the liver, a skill not possessed by most general surgeons. An aspect of some embodiments is the realization that a minimally invasive endovascular solution to this highly mortal condition is needed.

As noted above, an aspect of some embodiments is the realization that a challenge to successfully controlling injuries to the infrarenal IVC is that the drainage of the liver occurs through the hepatic veins which drain in the retrohepatic cava. Accordingly, some embodiments provide methods of placing a stent or endograft that can cover the entire retrohepatic cava without dire consequences including death from liver failure.

FIG. 13 illustrates an inferior vena cava 1200, as well as a liver 1202, a gallbladder 1204, a falciform ligament 1206 and various veins, including the right hepatic vein 1210, the middle hepatic vein 1212, a left hepatic vein 1214, and various short hepatic veins 1216.

FIGS. 14A and 15A illustrate the general structures illustrated in FIG. 11, except that the inferior vena cava 1200 has experienced a trauma or injury 1230, 1240. As illustrated in these figures, the trauma or injury 1230, 1240 can cause internal bleeding, which can necessitate the use of a stent or endograft in order to restore the inferior vena cava 1200 and reduce or stop the bleeding.

Accordingly, as illustrated in FIGS. 14A-15B, some embodiments provide for methods and apparatuses of placing a stent within the inferior vena cava 1200 to provide coverage or treatment of an injury 1230, 1240 while still allowing flow through the inferior vena cava 1200 to all, a majority, or a plurality of the right hepatic vein 1210, the middle hepatic vein 1212, a left hepatic vein 1214, and/or various short hepatic veins 1216.

For example, FIG. 14B illustrates that a stent or endograft 1250 has been placed within the inferior vena cava 1200. According to some embodiments, the surrounding vasculature of the inferior vena cava 1200, such as the right hepatic vein 1210, the middle hepatic vein 1212, a left hepatic vein 1214, and/or various short hepatic veins 1216 can be located using a delivery system having an imaging mechanism(s), such as that discussed above in FIGS. 5A-5F. Using the imaging mechanism, such as IVUS, the stent or endograft 1250 can be positioned such that an aperture 1252 of the stent or endograft 1250 can open towards the orifices of all, a majority, or a plurality of the right hepatic vein 1210, the middle hepatic vein 1212, and/or a left hepatic vein 1214. Thus, the stent or endograft 1250 can be placed within the inferior vena cava 1200 such that the traumatic injury 1230 is covered while an aperture or fenestration 1252 allows flow through the inferior vena cava 1200 to all, a majority, or a plurality of the hepatic veins extending from the inferior vena cava 1200.

FIGS. 15A-15B provide another illustration of the method shown in FIGS. 14A-14B. In FIGS. 15A-15B, the trauma or injury 1240 is located further down the inferior vena cava 1200, adjacent to the short hepatic veins 1216. Accordingly, using an embodiment of the delivery system and imaging mechanism(s) discussed herein, a stent or endograft 1260 can be positioned and expanded such that an aperture or fenestration 1262 of the stent or endograft 1260 opens to all, a majority, or a plurality of the short hepatic veins 1216.

In some embodiments, the stent or endograft 1250, 1260 can have a generally cylindrical, non-tapering shape. In some embodiments, the stent or endograft 1250, 1260 can have a diameter from about 15 mm to about 27 mm in size. For example, the diameter can be from about 18 mm to about 24 mm in size. Further, the diameter can be from about 20 mm to about 22 mm in size.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology. In particular, although the embodiments discussed herein have been discussed with reference to the inferior vena cava and specific vessels associated therewith, the methods and systems disclosed herein can be used with other vessels that have branch vessels extending therefrom. Indeed, some embodiments provide a versatile surgical delivery system that can be configured with at least one imaging mechanism arranged thereon in order to facilitate delivery, positioning, and/or deployment of an implant, such as a stent. For example, the imaging mechanism can comprise an ultrasound probe configured to emit an ultrasound beam within the vessel to determine the vessel geometry and facilitate desired placement of the stent.

There may be many other ways to implement the subject technology. Various functions and elements described herein may be accomplished differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1. A system for positioning a stent, the system comprising:
an elongate member;
an expandable stent coupled to the elongate member in a collapsed configuration, the stent having a sidewall aperture; and
an ultrasound probe positioned adjacent the aperture and configured to emit an ultrasound beam radially outwardly from the aperture while the stent is in the collapsed configuration.

2. The system of claim 1, wherein the probe is positioned radially within the stent.

3. The system of claim 2, wherein the probe is coupled to the elongate member.

4. The system of claim 1, wherein the elongate member comprises a catheter.

5. The system of claim 1, further comprising a sheath extending over the stent, and wherein the ultrasound probe is coupled to the sheath.

6. The system of claim 1, wherein the probe comprises a crystal element.

7. A system for positioning a stent, the system comprising:
an elongate member;
an expandable stent, coupled to the elongate member, having a distal end; and
an ultrasound probe coupled to the elongate member, positioned distal to the stent distal end, and configured to emit an ultrasound beam radially outwardly within a blood vessel.

8. The system of claim 7, wherein the stent is coupled to the elongate member in a collapsed configuration.

9. The system of claim 7, wherein the ultrasound probe is coupled to a dilator tip.

10. The system of claim 9, wherein the elongate member comprises the dilator tip.

11. The system of claim 7, wherein the ultrasound probe is configured to emit the beam in a radial direction.

12. The system of claim 7, wherein the ultrasound probe comprises a crystal element.

13. A method of implanting a stent in a first vessel, the method comprising:
advancing, into the first vessel, a delivery member carrying an expandable stent having an aperture formed in a sidewall of the stent;
imaging the first vessel with an ultrasound probe coupled to the delivery member to locate a second vessel;
positioning the stent such that the aperture is longitudinally and rotationally aligned with an orifice of the second vessel; and
expanding the stent such that the aperture opens to the orifice.

14. The method of claim 13, wherein the first vessel forms a junction with the second vessel, and the method further comprises:
positioning a second expandable stent at the junction, the second stent having first and second sections, a sidewall, and a second aperture formed in the sidewall; and
expanding the second stent at the junction, such that (1) the second stent first section extends through the aperture and into the second vessel, (2) the second aperture opens to the primary vessel, and (3) the second section is positioned within the primary vessel.

15. The method of claim 14, wherein the second stent is positioned such that the second aperture is aligned with a lumen of the stent, to maintain fluid communication through the stent.

16. The method of claim 14, wherein the junction comprises a venous confluence.

17. The method of claim 14, wherein the junction comprises an arterial bifurcation.

18. The method of claim 13, wherein the imaging comprises locating a plurality of second vessels, the plurality of second vessels comprising a plurality of orifices, and wherein the expanding comprises orienting the stent such that the (i) the aperture opens to the plurality of orifices and (ii) a first portion of the stent extends along a traumatic injury of the first vessel.

19. The method of claim 13, wherein the first vessel is an inferior or superior vena cava.

20. The method of claim 13, wherein the second vessel orifice opens to a hepatic vein.
